(19) United States
(12) Patent Application Publication
(10) Pub. No.: US 2025/0325754 A1
(43) Pub. Date: Oct. 23, 2025

(54) PLUNGER SPEED CONTROL INJECTION SYSTEM

(71) Applicant: NOBLE INTERNATIONAL, LLC, Orlando, FL (US)

(72) Inventor: Brian MAXFIELD, Orlando, FL (US)

(21) Appl. No.: 19/121,977

(22) PCT Filed: Oct. 18, 2023
(86) PCT No.: PCT/US2023/035381

§ 371 Date: Apr. 17, 2025

Related U.S. Application Data
(60) Provisional application No. 63/417,438, filed on Oct. 19, 2022.

(51) Int. Cl. A61M 5/315; A61M 5/32

(52) U.S. Cl. CPC A61M 5/31501; A61M 5/31511; A61M 5/3257

(57) ABSTRACT

In embodiments described herein are methods and devices including an injection device having a housing, a plunger, a safety shield movable proximally and distally, and a syringe for receiving the plunger, said syringe comprising a syringe ramped portion. In some embodiments, the device include a plunger control system including a plunger brake, a lock ring, a slider, a clutch, and a biasing member for controlling movement of the plunger.

### BACKGROUND

Many medications are taken into the body by way of an injection. An injection device typically includes a housing, a plunger and a needle. When a force is applied to a plunger by a mechanical means, such as in an auto injector or manually by the person administering the injection, the medicament is delivered through the needle into the target area of the subject. A resistance is caused by delivery of a fluid medicament from an injection device. The amount of time required to provide an injection is dictated, in part, by the amount of resistance due to the delivery of the fluid medicament. Medicaments include different viscosities which also contributes to the amount of time required to perform an injection. Injection training devices are used to train users to deliver injections. There is often a fear associated with injecting a subject. Injection training devices are used to calm that fear by allowing a user to practice the injection process with a simulated injection device. With medications having different viscosities, and consequently, differing in terms of injection time, simulating the injection experience can be challenging. Moreover, with more medicaments becoming increasingly viscous, increasing delivery times, difficulty occurs in producing an injection training device for simulating a drug delivery device.

### BRIEF DESCRIPTION OF THE DRAWINGS

A more particular description briefly stated above will be rendered by reference to specific embodiments thereof that are illustrated in the appended drawings. Understanding that these drawings depict only typical embodiments and are not therefore to be considered to be limiting of its scope, the embodiments will be described and explained with additional specificity and detail through the use of the accompanying drawings in which:

FIG. 1 includes a side view of an injection device embodiment.

FIG. 2 shows a partial cross-sectional view of an embodiment of an injection device.

FIG. 3 shows a partial cross-sectional view of an embodiment of an injection device.

FIG. 4 shows a partial cross-sectional view of an embodiment of an injection device.

FIG. 5 shows a partial cross-sectional view of an embodiment of an injection device.

FIG. 6 shows a partial cross-sectional view of an embodiment of an injection device.

FIGS. 7A-C show a partial cross-sectional view of an embodiment of an injection device.

### DETAILED DESCRIPTION

For the purposes of promoting an understanding of the principles and operation of the invention, reference will now be made to the embodiments illustrated in the drawings and specific language will be used to describe the same. It will nevertheless be understood that no limitation of the scope of the invention is thereby intended, such alterations and further modifications in the illustrated device, and such further applications of the principles of the invention as illustrated therein being contemplated as would normally occur to those skilled in the art to which the invention pertains.

The terminology used herein is for the purpose of describing particular embodiments only and is not intended to be limiting. As used herein, the singular forms “a,” “an,” and “the” are intended to include the plural forms as well, unless the context clearly indicates otherwise these terms do not denote a limitation of quantity, but rather denote the presence of at least one of the referenced item. Furthermore, to the extent that the terms “including,” “includes,” “having,” “has,” “with,” or variants thereof are used in either the detailed description and/or the claims, such terms are intended to be inclusive in a manner similar to the term “comprising.” Moreover, unless specifically stated, any use of the terms first, second, etc., does not denote any order, quantity or importance, but rather the terms first, second, etc., are used to distinguish one element from another.

Notwithstanding that the numerical ranges and parameters setting forth the broad scope are approximations, the numerical values set forth in specific non-limiting examples are reported as precisely as possible. Any numerical value, however, inherently contains certain errors necessarily resulting from the standard deviation found in their respective testing measurements. Moreover, all ranges disclosed herein are to be understood to encompass any and all sub-ranges subsumed therein. As a non-limiting example, a range of “less than 10” can include any and all sub-ranges between (and including) the minimum value of zero and the maximum value of 10, that is, any and all sub-ranges having a minimum value of equal to or greater than zero and a maximum value of equal to or less than 10, e.g., 1 to 7.

The term “medicament” as used in describing the various embodiments of this invention includes an injectable liquid medicine, medication, drug, pharmaceutical, prescriptive, agent, antidote, anti-venom, hormone, stimulant, vasodilator, anesthetic, nutritional supplement, vitamin and/or mineral compound, saline solution, biological, organic compound, genetically and/or chemically modified protein and/or nucleic acids, or other liquid that is adapted to be injected into the tissue of a subject.

The term associated or association, as used herein, includes but is not limited to direct and indirect attachment, adjacent to, in contact with, partially or fully attached to, and/or in close proximity therewith. The term “in conjunction with” as used herein includes but is not limited to synchronously or near synchronous timing, the phrase may also include the timing of outputs, where one output directly follows another output.

As used herein, the terms “subject”, “user” and “patient” are used interchangeably. As used herein, the term “subject” refers to an animal, and most preferably a human.

The term “injection device” as used herein includes both medicament-containing devices and non-medicament containing devices. This term may refer to training devices and needle-containing injection devices.

Many of the injection devices on the market require patients to self-administer injections, for example, via a pre-filled syringe in a non-limiting example. Other injection devices used for self-administration may include an autoinjector, for example. Between injection devices, and between medicaments being injected, there is often a varying amount of force is required to deliver an injection. The inventors herein have discovered a device in which a plunger control mechanism may simulate the speed of a plunger or control the speed of the plunger in various embodiments. The embodiments presented herein avoid the need for damping fluid providing an opportunity for a lower cost functioning device and an easier assembly of the device. The injection time in the embodiments described herein is more controllable. In some instances this may occur by a spring force in lieu of relying on stopper friction as used in other devices. In the embodiments described herein, the plunger control system is superior to friction and linear damper based systems for at least the reasons described herein.

According to one embodiment, provided is an injection device that includes a housing; a plunger; a safety shield movable proximally and distally; a syringe for receiving the plunger, the syringe comprising a syringe ramped portion; and a plunger control system. The plunger control system includes a plunger brake for interfacing with the plunger to control movement of the plunger during use of the device; a lock ring axially rotatable relative to the plunger, such that upon actuation of the device by proximal movement of the safety shield, the lock ring is rotated in a first direction; a slider associated with the lock ring such that rotation of the lock ring moves the slider distally; a clutch interfacing with the slider such that distal movement of the slider moves the clutch distally, the clutch interfacing with the syringe, wherein the interface between the clutch and the syringe rotates the clutch to engage a syringe cap; and a biasing member, wherein actuation of the device by proximal movement of the safety shield actuates by compressing the biasing member moving the plunger brake distally, restricting plunger movement, and distal movement of the safety shield releases the biasing member releasing the plunger brake.

In a specific embodiment, actuation of the biasing member moves the plunger brake distally which in turn activates the plunger brake, such that the plunger brake interfaces with the syringe ramped portion controlling movement of the plunger. In a more specific embodiment, the interface between the plunger brake and the syringe ramped portion increases the force of the plunger brake on the plunger, reducing the speed of distal plunger movement.

In another specific embodiment, proximal movement of the safety shield rotates the lock ring. Rotation of the lock ring moves the clutch distally via one or more slider teeth on a distal portion of the slider and interfacing clutch teeth on a proximal surface of the clutch. Furthermore, in a specific example, distal movement of the clutch activates the biasing member, thereby activating the plunger brake. In a specific example, the clutch comprises one or more inner ribs for interfacing with one or more plunger rails to restrict rotational movement of the clutch. Additionally, distal movement of the plunger allows rotation and reset of the clutch and release of the plunger brake. When the plunger is moved distally during use of the device, the plunger rails traverse the clutch inner ribs removing the interface between the plunger rails and the clutch inner ribs, allowing the clutch to rotate and move proximally within the device to a reset position.

According to another embodiment, provided is a method for controlling the speed of a plunger in an injection device. The injection device includes a housing, a plunger movable proximally and distally relative to the housing, a safety shield movable proximally and distally relative to the housing, wherein proximal movement of the safety shield occurs upon a force on the distal end of the safety shield; and a plunger control system. The plunger control system includes a plunger brake for interfacing with the plunger to control movement of the plunger during use of the device. The method involves moving the safety shield proximally upon a force on its distal end, wherein this movement causes the plunger brake to interface with the plunger to control a speed of the plunger during its distal movement.

In a specific embodiment, release of the force on the distal end of the safety shield allows the safety shield to move distally relative to the housing, releasing the plunger brake and allowing proximal movement of the plunger for a subsequent use of the device. In a more specific embodiment, the injection device comprises a lock ring, a slider, and a clutch that interface with one another upon proximal movement of the safety shield to compress a biasing member and activate the plunger break to control the speed of distal movement of the plunger. In a specific example, the method involves actuating the injection device, wherein proximal movement of the safety shield rotates the lock ring in a first direction, forcing the slider and the clutch in a distal direction. Conversely, distal movement of the clutch biases the biasing member, thereby moving the plunger brake in a distal direction, and causing a force on the plunger to decrease the speed of distal plunger movement. In a specific embodiment, the method involves removing the force on the distal end of the safety shield, which releases the safety shield in a distal direction, thereby releasing the biasing member, and allowing rotation of the lock ring in a second direction to a pre-use position. In a specific example, interfacing between the plunger brake and the plunger decreases a speed of plunger movement.

### Description of Illustrated Embodiments

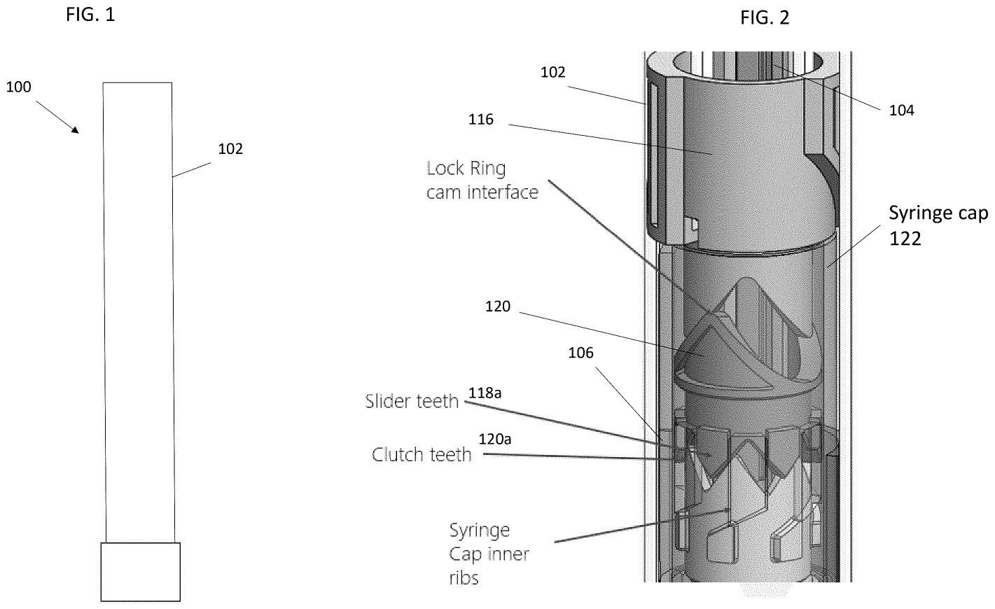

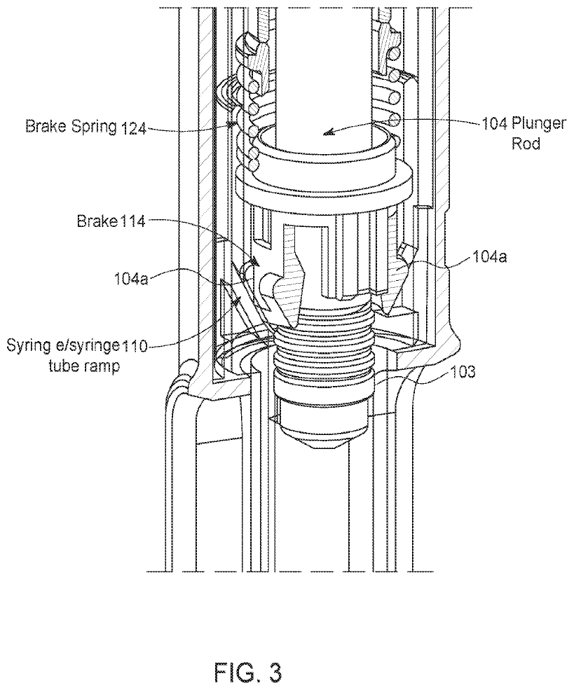

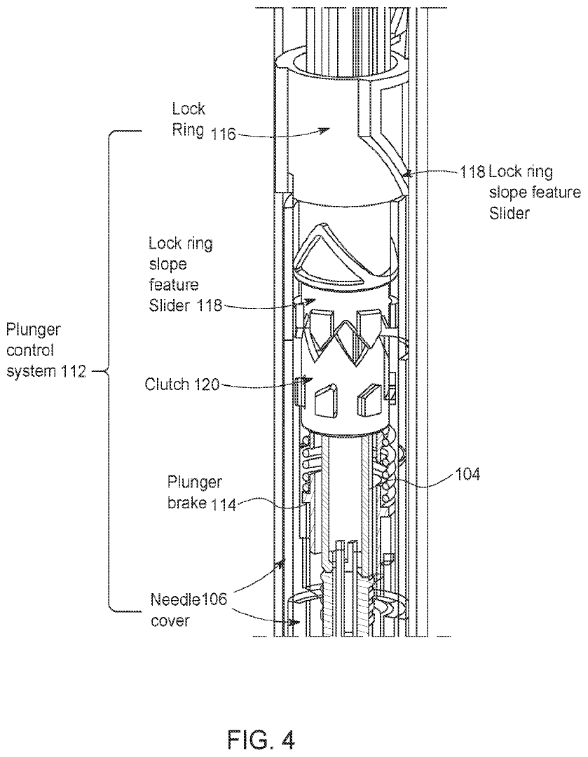

In a first embodiment as shown in the perspective and cross-sectional views of FIGS. 1-2 is an injection device 100 including a housing 102, a plunger 104, and a safety shield 106. The safety shield 106 is moved proximally and distally relative to the housing 102. The safety shield 106 extends from a distal portion of the housing 102 (i.e., a needle or injection simulation member in some instances) prior to actuation of the device 100. By a force on a distal portion of the safety shield 106, the device 100 may be actuated, and moved proximally relative to the housing 102. The device 100 includes a syringe 108 for receiving the plunger 104. The syringe 108 includes a syringe ramped portion 110 as shown in FIG. 3. The device 100 further includes a plunger control system 112 shown in FIG. 4. The plunger control system 112 controls the speed of the plunger 104. The plunger control system may include a plunger brake 114 for interfacing with the plunger 104 to control movement of the plunger 104 during use of the device 100. The plunger control system 112 may further include a lock ring 116 axially rotatable relative to the plunger 104, such that upon actuation of the device 100 by proximal movement of the safety shield 106, the lock ring 116 is rotated in a first direction relative to the housing 102. The plunger control system 112 may further include a slider 118 associated with the lock ring 116 such that rotation of the lock ring 116 moves the slider distally relative to the housing 102.

The plunger control system 112 may further include a clutch 120 for interfacing with the slider 118 such that distal movement of the slider 118 moves the clutch 120 distally relative to the housing 102, the clutch 120 interfacing with the syringe 108, wherein the interface between the clutch 120 and the syringe 108 rotates the clutch 120 to engage a syringe cap 122. The plunger control system 112 includes a biasing member 124, wherein actuation of the device 100 by proximal movement of the safety shield 106 actuates the device 100 by compressing the biasing member 124, moving the plunger brake 114 distally, restricting plunger movement by applying a pressure onto the plunger 104 to slow plunger movement. Distal movement of the safety shield 106 relative to the housing 102 releases the biasing member 124 releasing the plunger brake 114. Distal movement of the safety shield 106 may occur by release of the force on the distal end of the safety shield 106. In one embodiment, as shown in FIG. 3, during operation, actuation of the biasing member 124 moves the plunger brake 114 in a distal direction relative to the housing 102, activating the plunger brake 114, such that the plunger brake 114 interfaces with the syringe ramped portion 110, causing an increased force on the plunger 104 by the plunger brake 114, controlling movement of the plunger 104. When the plunger brake 114 is activated, it interfaces with the syringe ramped portion 110 which increases the contact between the plunger brake 114 and the plunger 104 (or increases a force on the plunger 104 via the plunger brake 114) to slow the movement/reduce the speed of distal plunger 104 movement. In some embodiments, the plunger brake 114 may include fingerlike projections 114a, which may interface with the ramped portion 110 to grip the plunger 104 and/or the stopper 103 of the device 100.

A force on the distal end of the safety shield 106 causes the safety shield 106 to move in a proximal direction relative to the housing 102 to actuate the device 100. During activation of the device 100, proximal movement of the safety shield 106 by a force on its distal end rotates the lock ring 116. Rotation of the lock ring 116 moves the clutch 120 distally via one or more slider teeth 118a on a distal portion of the slider and interfacing clutch teeth 120a on a proximal surface of the clutch 120 as shown in FIG. 2. Distal movement of the clutch 120 in this manner activates the biasing member 124, activating the plunger brake 114 as shown in FIG. 3.

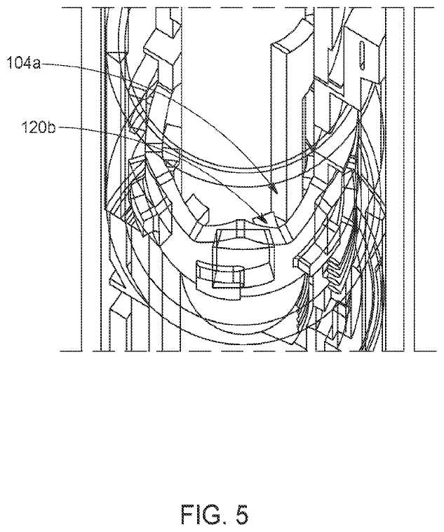

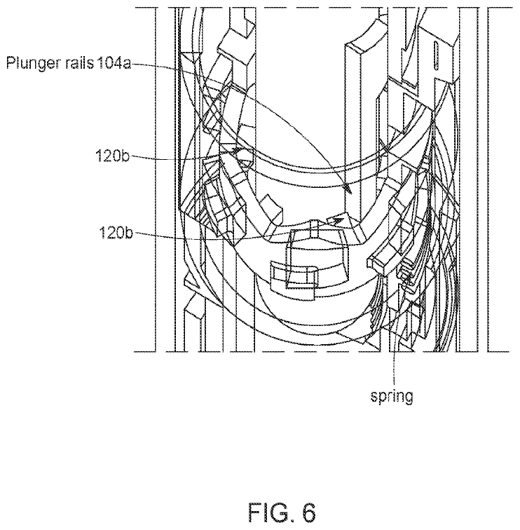

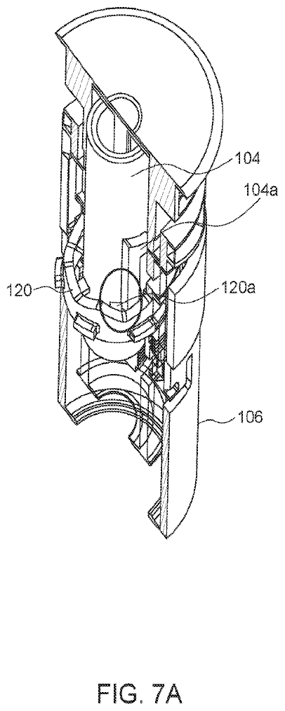

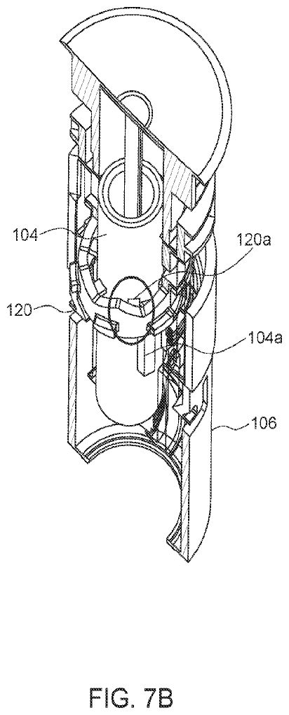

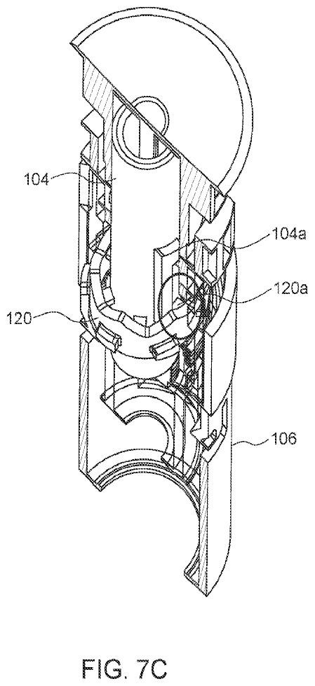

The clutch 120 includes one or more inner ribs 120b as shown in FIGS. 5-6 for interfacing with one or more plunger rails 104a on an outer surface of the plunger 104 to restrict rotational movement of the clutch 120. Continued distal movement of the plunger 104 allows rotation and reset of the clutch 120 and release of the plunger brake 114. Upon completion of the plunger 104 travel, the plunger rails 104a have moved past the inner ribs 120b of the clutch, and the clutch 120 is free to rotate and move in a proximal direction relative to the housing 102 to a reset position, whereby the plunger brake 114 is released and reset. This process is shown in FIGS. 7A-7C. FIG. 7A shows restriction of the clutch 120 rotation by way of the plunger rail 104a. FIG. 7B shows further distal movement of the plunger 104 such that the plunger rail 104a no longer restricts rotation of the clutch 120. Lastly, FIG. 7C shows reset of the plunger 104 and the clutch 120 for a subsequent use.

When the plunger 104 is moved distally during use of the device 100, the plunger rails 104a traverse the clutch inner ribs 120b removing the interface between the plunger rails 104a and the clutch inner ribs 120b, allowing the clutch 120 to rotate and move proximally within the device to a reset position as shown in FIG. 7C.

In an embodiment herein, a method for controlling a speed of a plunger 104 in an injection device 100 is provided. The method includes a device 100 including a housing 102, a plunger 104, a safety shield 106 movable proximally and distally relative to the housing 102, wherein proximal movement of the safety shield 106 occurs upon a force on a distal end of the safety shield 106, and a plunger control system 112, including a plunger brake 114 for interfacing with the plunger 104 to control movement of the plunger 104 during use of the device 100. The method includes when the safety shield 106 is moved proximally upon a force on its distal end, the plunger brake 114 interfaces with the plunger 104 to control the speed of the plunger 104 during its distal movement. A release of the force on the distal end of the safety shield 106 allows the safety shield 106 to move distally relative to the device housing 102, releasing the plunger brake 114 and allowing retraction or proximal movement of the plunger 104 to reset the device 100 for a subsequent use. The device comprises a lock ring 116, a slider 118, and a clutch 120 that interface with one another upon proximal movement of the safety shield 106 to activate a biasing member 124, to activate the plunger break 114 and control the speed of movement of the plunger 104. In some non-limiting embodiments, activating the biasing member 124 includes compressing the biasing member 124. Actuation of the device 100 and proximal movement of the safety shield 106 rotates the lock ring 116 in a first direction, forcing the slider 118 and the clutch 120 in a distal direction. These components are reset back to their original positions, in one embodiment, upon release of a force on the distal end of the safety shield 106.

During use, distal movement of the clutch 120 biases the biasing member 124, moving the plunger brake 114 in a distal direction, causing a force on the plunger 104 to slow plunger movement. Removal of the force on the distal end of the safety shield 106 releases the safety shield 106, allowing it to extend in a distal direction relative to the housing 102, releasing the biasing member 124, and allowing rotation of the clutch 120 to a pre-use, reset position.

The embodiments described above refer to various combinations of elements. It is intended that variations of combinations of enumerated elements are part of this disclosure even if not explicitly described together.

What is claimed is:

1. An injection device, comprising:
a housing;
a plunger;
a safety shield movable proximally and distally;
a syringe for receiving the plunger, said syringe comprising a syringe ramped portion; and
a plunger control system, comprising
a plunger brake for interfacing with the plunger to control movement of the plunger during use of the device;
a lock ring axially rotatable relative to the plunger, such that upon actuation of the device by proximal movement of the safety shield, the lock ring is rotated in a first direction;
a slider associated with the lock ring such that rotation of the lock ring moves the slider distally;
a clutch interfacing with the slider such that distal movement of the slider moves the clutch distally, the clutch interfacing with the syringe, wherein the interface between the clutch and the syringe rotates the clutch to engage a syringe cap;
a biasing member, wherein actuation of the device by proximal movement of the safety shield actuates by compressing the biasing member moving the plunger brake distally, restricting plunger movement, and distal movement of the safety shield releases the biasing member releasing the plunger brake.

2. The device of claim 1 wherein actuation of the biasing member moves the plunger brake distally activating the plunger brake, such that the plunger brake interfaces with the syringe ramped portion controlling movement of the plunger.

3. The device of any of claim 1 or 2, wherein the interface between the plunger brake and the syringe ramped portion increases the force of the plunger brake on the plunger, reducing the speed of distal plunger movement.

4. The device of any of claims 1-3, wherein proximal movement of the safety shield rotates the lock ring.

5. The device of any of claims 1-4, wherein rotation of the lock ring moves the clutch distally via one or more slider teeth on a distal portion of the slider and interfacing clutch teeth on a proximal surface of the clutch.

6. The device of claim 5, wherein distal movement of the clutch activates the biasing member, activating the plunger brake.

7. The device of any of claims 1-5, wherein the clutch comprises one or more inner ribs for interfacing with one or more plunger rails to restrict rotational movement of the clutch.

8. The device of claim 6, wherein further distal movement of the plunger allows rotation and reset of the clutch and release of the plunger brake.

9. The device of claim 8, wherein when the plunger is moved distally during use of the device, the plunger rails traverse the clutch inner ribs removing the interface between the plunger rails and the clutch inner ribs, allowing the clutch to rotate and move proximally within the device to a reset position.

10. A method for controlling the speed of a plunger in an injection device comprising: a housing, a plunger movable proximally and distally relative to the housing, a safety shield movable proximally and distally relative to the housing, wherein proximal movement of the safety shield occurs upon a force on the distal end of the safety shield; and
a plunger control system, comprising:
a plunger brake for interfacing with the plunger to control movement of the plunger during use of the device;
wherein the method comprises moving the safety shield proximally upon a force applied on its distal end, thereby causing the plunger brake to interface with the plunger to control a speed of the plunger during its distal movement.

11. The method of claim 10, wherein release of the force on the distal end of the safety shield allows the safety shield to move distally relative to the housing, releasing the plunger brake and allowing proximal movement of the plunger for a subsequent use of the device.

12. The method of claim 10, wherein the device comprises a lock ring, a slider, and a clutch that interface with one another upon proximal movement of the safety shield to compress a biasing member and activate the plunger break to control the speed of distal movement of the plunger.

13. The method of claim 12, wherein actuation of the device and proximal movement of the safety shield rotates the lock ring in a first direction, forcing the slider and the clutch in a distal direction.

14. The method of claim 13, wherein distal movement of the clutch biases the biasing member, moving the plunger brake in a distal direction, causing a force on the plunger to decrease the speed of distal plunger movement.

15. The method of claim 14, wherein removal of the force on the distal end of the safety shield releases the safety shield in a distal direction, releasing the biasing member, allowing rotation of the lock ring in a second direction to a pre-use position.

16. The method of claim 10, wherein an interface between the plunger brake and the plunger decreases a speed of plunger movement.
